(19) United States
(12) Patent Application Publication
(10) Pub. No.: US 2022/0257809 A1
(43) Pub. Date: Aug. 18, 2022

(54) POLYMER PACKAGING AND USE THEREOF FOR PRESERVING A PHARMACEUTICAL COMPOSITION

(71) Applicant: Ceva Santé Animale S.A., Libourne (FR)

(72) Inventors: Sandrine Lacoste, Libourne (FR); Florence Guimberteau, Libourne (FR)

(21) Appl. No.: 17/621,951

(22) PCT Filed: Jun. 25, 2020
(86) PCT No.: PCT/FR2020/051112

§ 371 Date: Dec. 22, 2021

(30) Foreign Application Priority Data
Jun. 26, 2019 (FR) 1906965

(51) Int. Cl. A61L 2/08; A61J 1/14; B32B 27/32; B32B 27/30; B32B 27/08; B32B 7/12

(52) U.S. Cl. CPC A61L 2/081; B32B 2307/71; B32B 27/32; B32B 27/306; B32B 27/08; B32B 7/12; A61L 2202/181; A61L 2202/21; B32B 2439/80; B32B 2307/732; B32B 2307/7242; B32B 2250/246; B32B 2307/306; B32B 2439/60; B32B 2307/4026; A61J 1/1468

(57) ABSTRACT

The present invention concerns packaging for preserving a sterile pharmaceutical composition, comprising at least an inner layer of polypropylene having a thickness of between 200 and 800 μm and an outer layer made from polyethylene, the outer layer being in contact with the environment and having a thickness of between 300 and 1000 μm. The invention also concerns a method for the sterile preservation of a pharmaceutical composition using such packaging.

### OBJECT OF THE INVENTION

The present invention relates to the field of polymers, and in particular that of polymeric packaging, such as vials, which can be used for preserving sterile pharmaceutical compositions.

### TECHNICAL BACKGROUND OF THE INVENTION

Sterilization is an operation which is very important, indeed essential, in pharmaceutical composition manufacturing processes. Indeed, most pharmaceutical compositions have to be manufactured and preserved in sterile conditions before being administered to the patient, in order to prevent introduction of pathogenic germs, and to preserve an optimal chemical and physical stability. The sterilization step, in a method for sterile preservation of a pharmaceutical composition, can be carried out either on the packaging/pharmaceutical composition assembly or on each of them taken in isolation, followed by filling of the packaging with the pharmaceutical composition, all in a sterile environment.

The European Medicines Agency (EMA) has recently published new guidelines (EMA/CHMP/CVMP/QWP/850374/2015) on the conditions to be met for the sterilization of medical and veterinary products, including pharmaceutical compositions (active substances and excipients), but also the packaging containing said pharmaceutical compositions. As far as possible, these guidelines recommend carrying out the final sterilization step of the pharmaceutical composition, when present, in the final packaging thereof.

There are different types of methods which are commonly used for sterilizing an empty packaging, i.e. without a pharmaceutical composition inside, or a packaging containing a pharmaceutical composition. For example, the methods of sterilization by means of moist or dry heat, methods by means of irradiation, and methods by means of gaseous sterilization, which must be implemented last, when the heat or irradiation methods are not suitable, can be cited. The guidelines recommend cycles of several minutes, carried out at temperatures of between 110 and 121° C., for the methods of sterilization by means of moist heat. For performing dry heat sterilization methods, a period of at least two hours at a minimum temperature of 160° C. is recommended. Regarding the irradiation sterilization methods, the reference irradiation dose is at least 25 kGy.

These different sterilization methods thus require particular conditions for obtaining an optimal sterile pharmaceutical composition, which conditions the packaging containing the pharmaceutical composition must withstand successfully. More specifically, the packaging must exhibit properties of resistance, stability, mechanical resistance, or sturdiness, transparency, impermeability to environmental factors (light, oxygen, temperature), during and after the sterilization process, in particular so as to be able to preserve a sterile pharmaceutical composition for as long as possible. Said acceptable pharmaceutical packaging likewise may not itself alter the quality of the pharmaceutical compositions which it contains. Vice versa, the pharmaceutical composition may not alter the quality of said packaging.

There is thus currently a need to develop and provide new packaging which is resistant to increasingly harsh or demanding sterilization conditions, such as those recommended by the new EMA guidelines, and which makes it possible to preserve stable and sterile pharmaceutical compositions in a lasting manner.

### SUMMARY OF THE INVENTION

Faced with this problem, the inventors are proposing a novel resistant packaging for the preservation or storage of a sterile pharmaceutical composition. Said packaging in particular comprises a particular polymeric outer layer which makes it possible for said packaging to withstand the sterilization conditions recommended by the EMA guidelines, while preserving its sturdiness. More specifically, the inventors have surprisingly shown that a packaging comprising at least one polymeric outer layer of polyethylene of a thickness of between 300 and 1000 μm preserved its entire resistance when it was subjected to high doses of irradiation (around 40 kGy), thus allowing for lasting preservation of a sterile pharmaceutical composition.

The present invention thus relates to packaging, in particular for preserving a sterile pharmaceutical composition, comprising at least one inner layer of polypropylene having a thickness of between 200 and 800 μm and an outer layer made from polyethylene, the outer layer being in contact with the environment and having a thickness of between 300 and 1000 μm.

According to a particular embodiment, the inner layer of polypropylene has a thickness of between 300 and 700 μm, between 350 and 650 μm, between 400 and 600 μm, preferably between 450 and 550 μm, and even more preferably approximately 500 μm. According to another particular embodiment, the outer layer of polyethylene has a thickness of between 400 and 800 μm, between 450 and 750 μm, between 500 and 700 μm, preferably between 550 and 650 μm, and even more preferably approximately 600 μm. According to a preferred embodiment, the outer layer of polyethylene further comprises at least one anti-UV colorant.

According to another aspect of the invention, the packaging further comprises a central layer of gas barrier agent that is located between the inner layer of polypropylene and the outer layer of polyethylene, and having a thickness of between 10 and 250 μm, between 10 and 200 μm, between 20 and 80 μm, preferably between 20 and 50 μm, and even more preferably approximately 30 or 50 μm. The central layer of gas barrier agent preferably comprises ethylene-vinyl alcohol.

A particular packaging of the invention also comprises at least one of the following layers:
an inner layer of polypropylene having a thickness of approximately 500 μm,
a central layer comprising ethylene-vinyl alcohol and having a thickness of approximately 30 or 50 μm, and
an outer layer of polyethylene having a thickness of approximately 600 μm.

Said packaging preferably comprises two adhesive separator layers each having a thickness of between 5 and 50 μm, preferably approximately 10 μm, said two adhesive separator layers being located, respectively, between the inner layer of polypropylene and the central layer of gas barrier agent, and between the outer layer of polyethylene and the central layer of gas barrier agent.

According to a preferred embodiment of the invention, the polyethylene is a low-density polyethylene.

The present invention also relates to a packaging as described in the present application, further comprising a sterile pharmaceutical composition. The sterile pharmaceutical composition is preferably an aqueous or non-aqueous composition. According to a preferred embodiment, the non-aqueous composition is an oily composition. In particular, the sterile pharmaceutical composition comprises an active ingredient selected from amoxicillin, buserelin, toltrazuril, diclazuril, or cefalexin.

According to a preferred embodiment of the invention, the packaging is a bottle or a vial.

The invention also relates to a use of a packaging as described in the present application, for preserving a sterile pharmaceutical composition.

The invention also relates to a method for the sterile preservation of a pharmaceutical composition, comprising the following steps:

a) adding a pharmaceutical composition into packaging as described in the present application; and

b) sterilizing the packaging containing the pharmaceutical composition. According to a preferred embodiment, the packaging containing the pharmaceutical composition in step b) is sterilized by gamma irradiation, preferably at a dosage of between 15 and 40 kGy.

### DETAILED DESCRIPTION OF THE INVENTION

The present invention provides packaging that makes it possible to preserve a sterile pharmaceutical composition in the long-term. More particularly, said packaging has the advantage of being resistant to and stable under the sterilization conditions, in particular high doses of gamma irradiation, of being able to be impermeable to oxygen and to water, and of preserving its transparency.

The term “approximately,” as used in the present application, will be understood by a person skilled in the art, and can vary to a certain extent, depending on the context in which it is used. If some uses of this term are not clear to a person skilled in the art, depending on the context “approximately” means plus/minus 20%, preferably plus/minus 10%, of the value specified.

Packaging

The present invention relates to packaging, in particular for preserving a sterile pharmaceutical composition, comprising at least one inner layer of polypropylene having a thickness of between 200 and 800 μm and an outer layer made from polyethylene, the outer layer being in contact with the environment and having a thickness of between 300 and 1000 μm.

According to the invention, the packaging comprises at least one inner layer of polypropylene.

“Inner layer” means the layer located inside the packaging. It thus corresponds to the layer in contact with the pharmaceutical composition when this is present in the packaging.

The polypropylene (or polypropene) polymer, also referred to in the present application by the initials “PP,” has the chemical formula —(CH₂—CH(CH₃))ₙ—. The polypropylene results from the coordinative polymerization of propylene monomers (CH₂═CH—CH₃) in the presence of catalysts such as those used in the Ziegler-Natta method. The polypropylene has a density of approximately 0.9 g/cm³, more precisely between 0.895 and 0.920 g/cm³, and a Young's modulus, also referred to as the elastic modulus or traction, of between 1.1 and 1.6 Gpa (gigapascal). There are three different types of polypropylene. The first type comprises only propylene monomers in a solid semi-crystalline form, and is referred to as a “homopolymer of polypropylene.” The second type corresponds to random propylene/ethylene copolymers. They comprise, in addition to propylene monomers, a molar amount of 1 to 8% ethylene monomers. Finally, the third type corresponds to a homopolymer of polypropylene, further comprising a phase of random propylene/ethylene copolymers having a molar amount of 45 to 65% ethylene monomers. These are referred to as polypropylene impact copolymers. Commercially available polypropylenes are for example the polypropylene Aceso® PPM R021 by Total, and the polypropylene BORMED SB815MO by Borealis. According to a preferred embodiment of the invention, the polypropylene forming the inner layer of the packaging is a random propylene/ethylene copolymer comprising a molar amount of 3% ethylene monomers.

According to the invention, the inner layer of the packaging made of polypropylene has a thickness of between 200 and 800 μm. More particularly, the inner layer of polypropylene has a thickness of between 300 and 700 μm, between 350 and 650 μm, between 400 and 600 μm, 420 and 580 μm, between 430 and 570 μm, between 440 and 560 μm, between 450 and 550 μm, between 460 and 540 μm, between 470 and 530 μm, between 480 and 520 μm, between 490 and 510 μm, and preferably approximately 500 μm.

According to the invention, the packaging comprises at least one outer layer of polyethylene.

“Outer layer” means the layer located at the periphery of the [GH1][AH2] packaging. It thus corresponds to the layer in contact with the environment.

The polyethylene, also referred to in the present application by the initials “PE,” has the chemical formula —(CH₂—CH₂)—. It results from the radical polymerization of ethylene monomers (CH₂═CH₂) under very high pressure (1800 to 3000 bar) and at temperatures of around 200° C. Various types of polyethylenes exist, including low-density polyethylenes (“LDPE”), linear low-density polyethylenes (“LLDPE”), medium-density polyethylenes (“MDPE”), and high-density polyethylenes (“HDPE”).

As its name indicates, the LDPE has a density close to 0.92 g/cm³, more precisely between 0.895 and 0.925 g/cm³, less than the density of medium-density polyethylene (MDPE) (0.926-0.940 g/cm³) and of high-density polyethylene (HDPE) (0.941-0.965 g/cm³), which is explained by its branching rate which is higher than that of MDPE and of HDPE. The LDPE has a Young's modulus of between 0.1 and 0.3 Gpa. The LDPE may be of petroleum or fossil origin, or of vegetable origin. LDPEs of fossil origin are, for example, the LDPE “Bormed™ LE6607-PH” marketed by the company Borealis, and the LDPE “PureII PE 3020 D” marketed by the company LyondeIIBaseII. The MDPE has a Young's modulus of from 172 to 379 Mpa. Commercially available MDPEs are for example the MDPEs of the range Lumicene®, marketed by the company Total. The HDPE has a Young's modulus of between 0.5 and 1.2 Gpa. The HDPE may be either of petroleum origin, or of vegetable origin. An HDPE of vegetable origin, or which is “biosourced,” like all biosourced materials, has the effect of reducing the ecological or environmental footprint. HDPEs of fossil origin are for example the HDPEs marketed by the company Sabic, such as HDPE BM6246LS. An HDPE of vegetable origin, or which is biosourced, is for example the LDPE “SGF 4950” marketed by the company Braskem.

According to a preferred embodiment of the invention, the polyethylene is a low-density polyethylene.

According to a particular embodiment of the invention, the low-density polyethylene is of vegetable origin. The LDPE is preferably produced from plants, such as sugar cane. The use of a “biosourced” LDPE of this kind in the outer layer of the packaging has the effect of reducing the ecological or environmental footprint of the packaging, compared with the use of an LDPE of fossil origin. A particular LDPE that is “biosourced” or of vegetable origin is for example the LDPE “STN7006” marketed by the company Braskem.

According to the invention, the outer layer of the packaging made of PE has a thickness of between 400 and 800 μm. More particularly, the outer layer of PE has a thickness of between 450 and 750 μm, between 500 and 700 μm, between 550 and 650 μm, between 560 and 640 μm, between 570 and 630 μm, between 580 and 620 μm, between 590 and 610 μm, and preferably approximately 600 μm.

A preferred packaging of the invention for preserving a sterile pharmaceutical composition comprises at least one inner layer of polypropylene having a thickness of approximately 500 μm and an outer layer made from polyethylene, preferably low-density polyethylene, said outer layer being in contact with the environment and having a thickness of approximately 600 μm.

According to a particular embodiment, the outer layer of polyethylene, preferably of low-density polyethylene, of the packaging further comprises at least one additive. Said additives are for example antioxidants, plasticizers, stabilizers, lubricants, colorants (or “dye”), or mechanical strengtheners. The outer layer of polyethylene of the packaging preferably further comprises at least colorant, preferably having anti-UV properties. According to an even more preferred embodiment, the outer layer of polyethylene of the packaging comprises a colorant having anti-UV properties. Said packaging comprising an outer layer further comprising at least a or one anti-UV colorant is particularly suitable for the preservation of a sterile pharmaceutical composition that is sensitive to UV rays. According to a particular embodiment, the anti-UV colorant is present in a proportion of less than or equal to 5%, with respect to the total weight of the outer layer, in particular 5%, 4%, 3%, 2%, 1%, and preferably in a proportion of 3% by weight, with respect to the total weight of the outer layer. Colorants are, for example, the products of the Colorant Cleartint™ range marketed by the company Milliken, the products of the Mevopur® range marketed by the company Clariant, and the products marketed by the group Gabriel-Chemie, such as the reference HP 79860 UV.

According to a particular embodiment of the invention, the packaging further comprises a central layer of gas barrier agent. Said packaging comprising said central layer of gas barrier agent is particularly suitable for the preservation of a sterile pharmaceutical composition that is sensitive to gases, in particular sensitive to oxygen. “Central layer of gas barrier agent” means a central layer of gas barrier agent located between the inner layer of polypropylene and the outer layer of low-density polyethylene. On account of the position thereof, the central layer is thus neither in contact with the environment nor in contact with the sterile pharmaceutical composition when this is present in the packaging. The central layer of gas barrier agent preferably has a thickness of between 10 and 250 μm, between 10 and 200 μm, [GH3][AH4] between 20 and 80 μm, between 30 and 70 μm, preferably between 20 and 50 μm, and even more preferably approximately 30 or 50 μm. According to an even more particular embodiment of the invention, the packaging further comprises a central layer of gas barrier agent that is located between the inner layer of polypropylene and the outer layer of polyethylene, and having a thickness of between 10 and 250 μm, between 10 and 200 μm, between 20 and 80 μm, preferably between 20 and 50 μm, and even more preferably approximately 30 or 50 μm.

The central layer of gas barrier agent preferably comprises ethylene-vinyl alcohol or polyglycolic acid, advantageously ethylene-vinyl alcohol.

The ethylene-vinyl alcohol, also referred to in the present application by the initials “EVOH,” is a copolymer having the chemical formula —(CH₂—CHOH)ₓ—(CH₂—CH₂)_{y}. It results from the polymerization of ethylene monomers and vinyl acetate monomers, followed by hydrolysis. The copolymer EVOH is defined by the amount of moles of ethylene (%). The lower the percentage of moles of ethylene, the better the gas barrier agent properties. According to a preferred embodiment, the EVOH comprises between 20 and 60%, between 27 and 47%, and even more preferably approximately 32%, moles of ethylene. The products EVAL F101 and SOARNOL, marketed by the companies KURARAY and GOSHEI, can be cited as examples of EVOH.

The polyglycolic acid, also referred to in the present application by the initials “PGA,” is a polymer of vegetable origin, which is biodegradable and thermoplastic. The PGA in particular exhibits improved properties with respect to the gas barrier effect, heat resistance, moldability, transparency, and durability. An example of commercial PGA is the product KUREDUX by the company KUREHA.

Another preferred packaging of the invention, for preservation of a sterile pharmaceutical composition, comprises at least the following layers:
an inner layer of polypropylene having a thickness of approximately 500 μm,
a central layer comprising ethylene-vinyl alcohol and having a thickness of approximately 30 or 50 μm, and
an outer layer of polyethylene having a thickness of approximately 600 μm.

The packaging may optionally comprise one or more adhesive separator layers which can be located between the inner layer of PP and the outer layer of PE, or between the inner layer of PP and the central layer of gas barrier agent, and between the outer layer of PE and the central layer of gas barrier agent.

The adhesive separator layer comprises an adhesive agent formed from polymers, such as the polyethylenes, the polypropylenes, the EVOHs, the polyamides, the polycarbonates, the polystyrenes, the polyterephtalates, and which are modified by functional groups suitable for the support polymer. These include for example the commercial products of the trade mark ADMER® by the company Mitsui Chemical, which correspond to polypropylene or polyethylene modified with a maleic anhydride. A polyethylene modified with a maleic anhydride is for example the product ADMER™ NF408E.

In particular, the adhesive separator layer has a thickness of between 5 and 50 μm. More particularly, the adhesive separator adhesive separator layer has a thickness of between 5 and 40 μm, between 5 and 30 μm, between 5 and 20 μm, between 5 and 15 μm, and preferably of approximately 10 μm.

According to a preferred embodiment of the invention, the packaging comprises two adhesive separator layers each having a thickness of between 5 and 50 μm, preferably approximately 10 μm, said two adhesive separator layers being located, respectively, between the inner layer of polypropylene and the central layer of gas barrier agent, and between the outer layer of polyethylene and the central layer of gas barrier agent.

Within the context of the present application, an adhesive layer is also represented by the sign “/.” A packaging according to the invention comprising at least one adhesive layer can thus be represented in the following manners, depending on whether or not the central layer of gas barrier agent comprising ethylene-vinyl alcohol is present: PP/PE, and PP/EVOH/PE.

Another preferred packaging of the invention, for preservation of a sterile pharmaceutical composition, comprises at least the following layers:
an inner layer of polypropylene having a thickness of approximately 500 μm,
an adhesive separator layer having a thickness of approximately 10 μm, and
an outer layer of polyethylene having a thickness of approximately 600 μm.

Another preferred packaging of the invention, for preservation of a sterile pharmaceutical composition, comprises at least the following layers:
an inner layer of polypropylene having a thickness of approximately 500 μm,
an adhesive separator layer having a thickness of approximately 10 μm,
a central layer comprising ethylene-vinyl alcohol and having a thickness of approximately 30 μm,
an adhesive separator layer having a thickness of approximately 10 μm, and
an outer layer of low-density polyethylene having a thickness of approximately 600 μm.

Said preferred packaging is represented in the following manner: “PP (500 μm)/EVOH (30 μm)/LDPE (600 μm)” in the following examples.

Another preferred packaging of the invention, for preservation of a sterile pharmaceutical composition, comprises at least the following layers:
an inner layer of polypropylene having a thickness of approximately 500 μm,
an adhesive separator layer having a thickness of approximately 10 μm,
a central layer comprising ethylene-vinyl alcohol and having a thickness of approximately 50 μm,
an adhesive separator layer having a thickness of approximately 10 μm, and
an outer layer of low-density polyethylene having a thickness of approximately 600 μm.

Said preferred packaging is represented in the following manner: “PP (500 μm)/EVOH (50 μm)/LDPE (600 μm).”

Composition

Another aspect of the invention relates to a packaging as described in the present application, further comprising a sterile pharmaceutical composition.

“Pharmaceutical composition” means any pharmaceutical composition comprising an active ingredient. A pharmaceutical composition also includes veterinary compositions. According to a preferred embodiment of the invention, the pharmaceutical composition is a veterinary composition.

An active ingredient comprises, in a general manner, therapeutic and pharmaceutical agents, prophylactic agents, diagnostic agents, and any other agent capable of preventing and/or treating or diagnosing a pathology, an infection, a disease in humans or animals. More particularly, an active ingredient includes antibiotics, in particular the β-lactams such as amoxicillin, cephalosporins such as cefalexin, and tetracyclines such as oxytetracyclines, anti-infective agents, anticoccidial agents such as diclazuril and toltrazuril, vaccines such as vaccines comprising for example antibodies, antibody fragments, peptides, proteins, an interferon, an interleukin, a cytokine, and vitamins, non-steroid anti-inflammatory agents such as meloxicam and indomethacin, anthelmintic and antiparasitic agents such as praziquantel, pyrantel, ivermectin, permethrin, fipronil, and dinotefuran, insect growth regulators, antiviral agents, cardiotonic agents such as digoxin, hypertensive agents, diuretic agents, therapeutic agents for the treatment of heart failure such as spironolactone and inhibitors of the angiotensin-converting enzyme such as benazepril, induction and synchronization agents of estrous and ovulation such as buserelin. An active ingredient also includes diagnostic agents which can be polypeptides, nucleic acids, polysaccharides, lipids, glycoproteins, glycolipids, carbohydrates, agents allowing for marking certain tissues, such as agents containing radioisotopes, magnetic markers, fluorescent markers, chemiluminescent markers, and enzymatic markers.

The sterile pharmaceutical composition preferably comprises an active ingredient selected from amoxicillin, buserelin, toltrazuril, diclazuril, or cefalexin.

“Sterile pharmaceutical composition” means any pharmaceutical composition described above which has been used in a sterilization method aiming to destroy all microbial germs. These sterilization methods include in particular dry heat or moist heat (autoclaving) heat treatments, chemical treatments such as treatments using ethylene oxide, and treatments using UV radiation or ionizing radiation such as by exposure to gamma or beta radiation or to an accelerated electron beam.

The sterile pharmaceutical composition may be present in any form. For example, the sterile pharmaceutical composition may be a liquid composition, in particular an aqueous or non-aqueous composition. An example of a non-aqueous composition may be an oily composition. The sterile pharmaceutical composition may also be a solid composition such as a powder, a compressed tablet, a capsule, a granule, a sugar-coated pill, a hard capsule, a wafer capsule, a pill, a tablet, or a cream. The sterile pharmaceutical composition is preferably an aqueous or non-aqueous composition. Even more preferably, the non-aqueous sterile pharmaceutical composition is an oily composition.

According to the invention, the packaging is suitable for preserving a sterile pharmaceutical composition. According to a particular embodiment, the packaging has a three-dimensional structure (3D structure). This particular embodiment having a 3D structure thus excludes packaging in the form of films, in particular monolayer and multilayer polymeric films. According to a preferred embodiment of the invention, the packaging is a bottle or a vial. According to an even more preferred embodiment of the invention, the packaging is a vial which may contain a volume of sterile pharmaceutical composition of 10 ml, 25 ml, 50 ml, 100 ml, 250 ml, 500 ml, 1 l, 2 l, preferably 50 ml, 100 ml, 250 ml or 500 ml.

The invention also relates to a use of a packaging as described in the present application, for preserving a sterile pharmaceutical composition.

“Preserving a sterile pharmaceutical composition in the packaging according to the invention” can mean retaining, over time, physical and chemical properties of the sterile pharmaceutical composition, but also those of the packaging. Indeed, a deterioration of the packaging over time would at the same time alter the originally sterile pharmaceutical composition contained in said packaging. Thus, the packaging according to the invention makes it possible to preserve a sterile pharmaceutical composition in a lasting manner or over the long term. More specifically, the packaging according to the invention makes it possible to preserve a sterile pharmaceutical composition for at least 2, 3, 4, 5, 6, 7, 8, 9, 10, 11, 12, 15, 18, 24 months, 3 years, 4 years, 5 years, and more. The packaging according to the invention preferably makes it possible to preserve a sterile pharmaceutical composition for at least 6 months, preferably at least 12 months, 24 months, or 36 months.

Regarding the physical and chemical properties of the packaging, these are studied over time, by generally comparing them to glass packaging, which is the reference packaging routinely used by a person skilled in the art. The physical and chemical parameters of the packaging which are studied are, for example, parameters originating from differential scanning calorimetry which allows access to the thermal characteristics of the packaging associated with the change in physical state (melting, crystallization, glass transition) or chemical state (cross-linking, oxidation). It is also possible to study the mechanical properties of the vials by means of a simple drop test, as set out in the examples below.

Regarding the physical and chemical properties of the sterile pharmaceutical composition, these can be assessed in an absolute manner, and are governed by regulations or pharmacopoeias which define the physical and chemical parameters which have to be taken into account and indicate the extent to which said parameters may vary. The change in these parameters over time makes it possible to assess the physical and chemical stability of the sterile pharmaceutical compositions and, more generally, the preservation thereof over time. The parameters may be either qualitative or quantitative. The qualitative parameters which can be taken into account are in particular the color, the transparency and the smell of the composition. The quantitative parameters are, for example, the dosage or the titration of an active ingredient in the composition, the relative percentage of degradation product with respect to the active ingredient, the pH, and the viscosity. Generally, in the pharmaceutical field, the measurements of the titration of the active ingredient and of the relative percentage of degradation product with respect to the active ingredient are sufficient for determining the stability of the pharmaceutical composition over time. Thus, a composition is considered stable when the content of active ingredient in the composition, and the relative percentage of degradation products, vary in proportions such that the effectiveness and the safety of the composition are not significantly changed. The measurements can be made at different times, for example 3, 6, 12, 18 and 24 months, at different temperatures, for example 4° C., 25° C. and 40° C., and under different humidity conditions. More particularly, a composition can be considered stable when the variation in the content of active ingredient does not exceed 10%, preferably 5%. A composition can also be considered stable when the variation in the relative percentage of degradation products is at most 10%, preferably at most 5%. These ranges are recommended in particular by the European Pharmacopeia or described in the Guideline VICH GL3. More generally, it is considered that the preservation duration and the stability of a pharmaceutical composition will be longer, the smaller [GH5][AH6] the variation in the titer of the active ingredient over time, and the slower the emergence of degradation products.

Method

Another aspect of the invention relates to a method for the sterile preservation of a pharmaceutical composition. The sterile preservation of the composition may be achieved according to two particular embodiments.

In a first particular embodiment, a pharmaceutical composition is first added into packaging as described above. The packaging/pharmaceutical composition assembly is the sterilized by means of any method known to a person skilled in the art.

The invention thus relates to a method for the sterile preservation of a pharmaceutical composition, comprising the following steps:

a) adding a pharmaceutical composition into packaging as described above; and

b) sterilizing the packaging containing the pharmaceutical composition.

The method according to the invention thus allows for the sterilization and the sterile preservation of the pharmaceutical composition.

In a second particular embodiment, a pharmaceutical composition and a packaging as described above are sterilized separately. The sterile pharmaceutical composition is then [GH7][AH8] introduced into the sterile packaging, under sterile conditions.

The invention thus relates to a method for the sterile preservation of a pharmaceutical composition, comprising the following steps:

a) separately sterilizing a packaging as described above, and a pharmaceutical composition, so as to separately obtain a sterile packaging and a sterile pharmaceutical composition; and

b) adding said sterile pharmaceutical composition into said sterile packaging.

The methods described above for the sterile preservation of a pharmaceutical composition may further comprise an optional step c) of preservation of the packaging containing the sterile pharmaceutical composition, preferably under conditions suitable for good preservation of a pharmaceutical composition. For example, said step c) of preservation may be carried out protected from light, in a dry environment, at temperatures which do not exceed 20° C., under vacuum, it of course being possible for these different conditions to be combined with one another.

The step b) of sterilization, implemented in the methods described above, can be carried out by means of any sterilization method known to a person skilled in the art. In particular the step b) of sterilization may be carried out by means of moist heat, by means of dry heat, by means of gaseous sterilization, and by means of irradiation. According to a preferred embodiment, the step b) is carried out by means of irradiation, such as by means of gamma or beta irradiation, by means of electron beam, and preferably by means of gamma irradiation. According to an even more preferred embodiment, step b) is carried out by means of gamma irradiation at a dosage of between 15 and 40 kGy, or preferably at 15, 25, 40 kGy, and even more preferably at 25 or 40 kGy.

A preferred method of the invention for the sterile preservation of a pharmaceutical composition thus comprises the following steps:

a) adding a pharmaceutical composition into packaging as described above; and

b) sterilizing the packaging containing the pharmaceutical composition by means of gamma irradiation, preferably at a dosage of between 15 and 40 kGy.

Other aspects and advantages of the invention will appear from reading the following examples, which should be considered as illustrative and non-limiting.

### EXAMPLES

### Example 1: Drop Test

Protocol:

Samples of 20 vials of 100 ml were treated, or not treated, by gamma irradiation. The samples were then filled with water and underwent a drop test in the vertical position from a height of 1 meter and 40 centimeters, without or following irradiation. For the irradiated vials, the drop test was carried out just after irradiation treatment, or after several months of storage under vacuum and at ambient temperature.

Results:

The results for the different vial samples are set out in Table 1 below.

TABLE 1

| | | Number of vials |
|---|---|---|
| Composition of layers | | broken, out of 20 |
| of the vial | Irradiation conditions | (average data) |
| Vial according to the | no irradiation | 0 |
| invention: | 15-25 kGy | 0 |
| | 25 kGy | |
| | 15-25 kGy and 12 | 0 |
| | months of storage | |
| PP (500 μm)/EVOH | 25 kGy | 0 |
| (30 μm)/LDPE | 25 kGy and 12 | 0 |
| (600 μm) | months of storage | |
| | 40 kGy | 0 |
| | 40 kGy and 12 | 1 |
| | months of storage | |
| Vial according to the | no irradiation | 0 |
| invention: | 40 kGy | 0 |
| PP (480 μm)/EVOH | 40 kGy and 3 | 3 |
| (40 μm)/LDPE | months of storage | |
| (560 μm) | | |
| Vial according to the | no irradiation | 0 |
| invention: | 40 kGy | 0 |
| PP (470 μm)/EVOH | 40 kGy and 3 | 0 |
| (40 μm)/LDPE + DYE | months of storage | |
| (530 μm) | | |
| Vial according to the | no irradiation | 0 |
| invention: | 40 kGy | 0 |
| PP (490 μm)/EVOH | 40 kGy and 3 | 1 |
| (40 μm)/LDPE + DYE | months of storage | |
| (520 μm) | | |
| Comparison vial: | no irradiation | 0 |
| PP (500 μm)/EVOH | 15-25 kGy | 3 |
| (30 μm)/LDPE | 25 kGy | 15 |
| (200 μm) | 40 kGy | 17 |
| Comparison vial: | no irradiation | 0 |
| PP (600 μm)/EVOH | 40 kGy | 1 |
| (100 μm)/LDPE | 40 kGy and 12 | 13 |
| (250 μm) | months of storage | |

PP: polypropylene
EVOH: ethylene-vinyl alcohol
LDPE: low-density polyethylene

The results of Table 1 show that the vials of the invention comprising a polymeric outer layer of LDPE of an average thickness of 600 μm are resistant to different irradiation doses, up to 40 kGy and after 12 months of storage (just one vial out of twenty vial breaks under the most extreme conditions).

In contrast, most of the vials [GH9][AH10] comprising a thin LDPE layer of 200 μm break just after having been irradiated at 25 kGy (15/20) and at 40 kGy (17/20). Furthermore, a significant number of vials (13/20) comprising a thin LDPE layer of 250 μm breaks under the most extreme storage conditions (40 kGy irradiation and 12 months of storage).

These results show that a polymeric outer layer of PE [GH11][AH12] of a thickness of greater than 250 μm, in particular of between 300 and 1000 μm, is necessary for providing packaging that is resistant to high doses of irradiation, thus making it possible to preserve a sterile pharmaceutical composition over a long period (at least 12 months).

### Example 2: Study of the Gas and Water Barrier Effect

Water Permeability Test Protocol:

Irradiated or non-irradiated vials according to the invention were filled with 10 g of molecular sieve, then hermetically sealed, weighed, and placed in a climate chamber at 40° C./75% RH for approximately 3 months.

They were weighed regularly throughout this period in order to measure their weight increase, which makes it possible to assess the capacity of the packaging to allow for penetration of water vapor.

Oxygen Permeability Test Protocol:

The measurements of permeability to oxygen, on the irradiated or non-irradiated vials according to the invention, were carried out according to the standard ASTM F2622.

Results:

The results for the different vial samples are set out in Table 2 below.

TABLE 2

| | | Average water | |
|---|---|---|---|
| Composition of | | vapor transmission | |
| layers of the vial | | coefficient | Oxygen |
| according to the | Irradiation | (g/24 h at 40° | transmission |
| invention | conditions | C. + 75% RH) | (cm3/24 h) |
| PP (500 μm)/ | not irradiated | 0.0026 | 0.0013 |
| EVOH (30 μm)/ | 15 kGy | 0.0035 | — |
| LDPE (600 μm) | 25 kGy | 0.0035 | 0.0015 |
| | 40 kGy | 0.0032 | 0.0011 |
| PP (500 μm)/ | 25 kGy | ND | 0.0021 |
| EVOH (30 μm)/ | 40 kGy | ND | 0.0025 |
| LDPE (600 μm) | | | |
| Stored at 8 | | | |
| months after | | | |
| treatment | | | |
| PP (470 μm)/ | 40 kGy | 0.003 | ND |
| EVOH (30 μm)/ | | | |
| LDPE + DYE | | | |
| (530 μm) | | | |
| PP (490 μm)/ | not irradiated | ND | 0.002 |
| EVOH (40 μm)/ | 40 kGy | 0.0027 | 0.0020 |
| Purell 3020D | | | |
| LDPE + DYE | | | |
| (520 μm) | | | |

PP: polypropylene
EVOH: ethylene-vinyl alcohol
LDPE: low-density polyethylene
RH: relative humidity
AT: ambient temperature

ND: not determined

The results of Table 2 show that the gamma irradiation has no impact on the oxygen barrier properties or in the water vapor barrier properties of the packagings of the invention.

### Example 3: Study of the Stability of the Pharmaceutical Composition

Protocol:

The vials according to the invention (PP (500 μm)/EVOH (30 μm)/LDPE (600 μm) were filled with a suspension of amoxicillin, then irradiated by means of gamma irradiation at 4 different doses (7, 15, 25, and 40 kGy). Said vials were then analyzed before and after storage for 6 months at 40° C./75% RH.

Results:

The results for the different vial samples are set out in Table 3 below.

TABLE 3

| | | | Amoxicillin | |
|---|---|---|---|---|
| | | | dosage | |
| | | Amoxicillin | (mg/ml) | |
| | | dosage | after T6 | Dosage |
| | Appear- | (mg/ml) | months of | of total |
| Irradiation | ance of | prior to | storage at 40° | degradation |
| conditions | product | storage | C./75% RH | products (%) |
| Not | white cream | 200 | — | 1.2 |
| irradiated | suspension | | | |
| Not | white cream | 200 | 195 | 2.0 |
| irradiated | suspension | | | |
| 7 kGy | beige cream | 200 | 193 | 2.1 |
| | suspension | | | |
| 15 kGy | pale brown | 200 | 193 | 2.2 |
| | cream | | | |
| | suspension | | | |
| 25 kGy | pale brown | 200 | 193 | 2.3 |
| | cream | | | |
| | suspension | | | |
| 40 kGy | pale brown | 200 | 191 | 2.2 |
| | cream | | | |
| | suspension | | | |
| 7 kGy | beige cream | 200 | 195 | 2.2 |
| | suspension | | | |
| 15 kGy | pale brown | 200 | 195 | 2.2 |
| | cream | | | |
| | suspension | | | |
| 25 kGy | pale brown | 200 | 194 | 2.0 |
| | cream | | | |
| | suspension | | | |
| 40 kGy | pale brown | 200 | 193 | 2.1 |
| | cream | | | |
| | suspension | | | |

The first line of Table 3 corresponds to the dose of amoxicillin and degradation products at T0, i.e. without storage.

The results of Table 3 show that the step of irradiation, whatever the dose, does not impact the stability of the amoxicillin suspension.

Indeed, the active ingredient concentration remains in the acceptable range for a pharmaceutical product of +1-5%, and the amount of degradation products observed does not exceed what is acceptable.

The inventors have shown that the packaging according to the invention, following irradiation, made it possible to preserve the stability of a pharmaceutical product stored in accelerated conditions (40° C./75% RH) for 6 months. These results obtained under these accelerated conditions ensure stability of the pharmaceutical product of at least 18 months at ambient temperature.

1. A packaging for preserving a sterile pharmaceutical composition, comprising at least one inner layer of polypropylene having a thickness of between 200 and 800 μm and an outer layer made from polyethylene, the outer layer being in contact with the environment and having a thickness of between 300 and 1000 μm.

2. The packaging according to claim 1, wherein the inner layer of polypropylene has a thickness of between 300 and 700 between 350 and 650 between 400 and 600 preferably between 450 and 550 and even more preferably approximately 500 μm.

3. The packaging according to claim 1, wherein the outer layer of polyethylene has a thickness of between 400 and 800 between 450 and 750 between 500 and 700 preferably between 550 and 650 and even more preferably approximately 600 μm.

4. The packaging according to claim 1, wherein the outer layer of polyethylene further comprises at least one anti-UV colorant.

5. The packaging according to claim 1, wherein the packaging further comprises a central layer of a gas barrier agent that is located between the inner layer of polypropylene and the outer layer of polyethylene, and having a thickness of between 10 and 250 between 10 and 200 between 20 and 80 preferably between 20 and 50 and even more preferably approximately 30 or 50 μm.

6. The packaging according to claim 5, wherein the central layer of gas barrier agent comprises ethylene-vinyl alcohol.

7. The packaging according to claim 5, wherein the packaging comprises at least the following layers:
an inner layer of polypropylene having a thickness of approximately 500 μm,
a central layer comprising ethylene-vinyl alcohol and having a thickness of approximately 30 or 50 μm, and
an outer layer of polyethylene having a thickness of approximately 600 μm.

8. The packaging according to claim 5, wherein the packaging further comprises two adhesive separator layers each having a thickness of between 5 and 50 preferably approximately 10 said two adhesive separator layers being located, respectively, between the inner layer of polypropylene and the central layer of gas barrier agent, and between the outer layer of polyethylene and the central layer of gas barrier agent.

9. The packaging according to claim 1, wherein the polyethylene is a low-density polyethylene.

10. The packaging according to claim 1, further containing a sterile pharmaceutical composition.

11. The packaging according to claim 10, wherein the sterile pharmaceutical composition is an aqueous or non-aqueous composition.

12. The packaging according to claim 11, wherein the non-aqueous composition is an oily composition.

13. The packaging according to claim 1, wherein the sterile pharmaceutical composition comprises an active ingredient selected from amoxicillin, buserelin, toltrazuril, diclazuril, or cefalexin.

14. The packaging according to claim 1, wherein the packaging is a bottle or a vial.

15. Use of the packaging as defined according to claim 1 for preserving a sterile pharmaceutical composition.

16. A method for the sterile preservation of a pharmaceutical composition, comprising the following steps:
a) adding a pharmaceutical composition into packaging as defined according to claim 1; and
b) sterilizing the packaging containing the pharmaceutical composition.

17. The method according to claim 16, wherein the packaging containing the pharmaceutical composition in step b) is sterilized by gamma irradiation, preferably at a dosage of between 15 and 40 kGy.
